(19) United States
(12) Patent Application Publication
(10) Pub. No.: US 2015/0344545 A1
(43) Pub. Date: Dec. 3, 2015

(54) Method of Treating and Preventing Infectious Diseases Using Colostrum

(71) Applicants: Harry Leneau, Jasper, MO (US); Judith B. Leneau, Jasper, MO (US)

(72) Inventors: Harry Leneau, Jasper, MO (US); Judith B. Leneau, Jasper, MO (US)

(21) Appl. No.: 14/294,286

(22) Filed: Jun. 3, 2014

(51) Int. Cl. C07K 16/08

(52) U.S. Cl. CPC C07K 16/081; C07K 2317/76

(57) ABSTRACT

A method of treating and/or preventing infectious disease in mammals, such as canines, includes administering an effective amount of a colostral composition to a mammal which has been produced by a donor mammal and includes at least one type of antibody having a binding specificity for at least one pathogen. The at least one type of antibody is generated by the donor mammal in response to being challenged with at least one challenge material.

### TECHNICAL FIELD

The present disclosure relates to compositions and methods of providing treatment and/or protection against diseases, such as viral and bacterial infections, for mammals, such as canines, using colostrum based compositions.

### BACKGROUND

There are many bacterial and viral agents that afflict mammals with various types of disease and illness. One example of such an agent is parvovirus. There are many different strains of parvovirus with different strains being capable of afflicting different species of animals, including canines, felines, and even humans. Canine parvovirus is one of the most widespread and lethal diseases that can afflict canines. Canine parvovirus infection is a highly contagious viral illness that spreads easily through direct contact with an infected animal or through indirect contact with an infected animal's feces. The disease caused by this virus is much more common in puppies than in adult dogs because it thrives on the developing cells of the young animal.

Canine parvovirus manifests itself in two different forms. The more common form is the intestinal form, which is characterized by severe, bloody diarrhea, lethargy, anorexia, fever, vomiting, and severe weight loss. The intestinal form of canine parvovirus affects the body's ability to absorb nutrients, and an affected animal will quickly become dehydrated and weak from lack of protein and fluid absorption. The less common form is the cardiac form, which attacks the heart muscles of very young puppies, often leading to death.

There is presently no known and accepted cure for canine parvovirus. Once infected, canines are typically treated by administering fluids to prevent dehydration and dispensing medicine and antibiotics to combat infection. The survival rate of infected animals depends on how quickly the infection is diagnosed and the dog begins treatment. If the dog begins treatment within 24 hours of the onset of symptoms, it typically has a 50 percent chance of survival. However, even if the dog survives, it is still possible to contract the illness again.

Vaccines have been developed to prevent some of the most common and serious canine diseases, including canine parvovirus. Dogs typically receive vaccinations when they are puppies starting at 5 or 6 weeks of age. Vaccines can reduce the chances of a dog becoming infected with the virus. However, even when appropriately vaccinated, it is possible for a dog to become infected due to the extreme virulent and contagious nature of the virus. For example, previously known vaccines may be neutralized in the presence of maternally derived antibodies (MDA) which are present in the puppies system for a period of time after birth. The MDA may persist in the puppies system up to four months or more at levels sufficient to interfere with vaccination.

What is needed is a composition and method for treating mammals, such as canines, that can reliably prevent infectious diseases, such as parvovirus, and that is capable of neutralizing active parvovirus infections as well as other types of bacterial and viral infections.

### BRIEF DESCRIPTION OF THE DRAWINGS

FIG. 1 is a flowchart of a method of treating and/or preventing infectious diseases in canines using a colostral treatment composition in accordance with the present disclosure.

FIG. 2 is a flowchart of a method of processing colostrum from a donor animal to produce a colostral treatment composition.

### DETAILED DESCRIPTION

For the purposes of promoting an understanding of the principles of the disclosure, reference will now be made to the embodiments illustrated in the drawings and described in the following written specification. It is understood that no limitation to the scope of the disclosure is thereby intended. It is further understood that the present disclosure includes any alterations and modifications to the illustrated embodiments and includes further applications of the principles of the disclosure as would normally occur to a person of ordinary skill in the art to which this disclosure pertains.

This disclosure is directed to compositions and methods for treating and/or preventing diseases in mammals caused by pathogens, such as viral agents, bacterial agents, and toxins.

The compositions and methods are based on the use of colostrum collected from donor animals, such as goats or cows, and administered to the mammal to be treated immediately after birth and prior to infection as a preventative measure, at the onset of any signs of infection to neutralize infection, and after infection to prevent relapse of infection. The donor animal is challenged at suitable times during pregnancy with one or more challenge materials that are selected to produce a certain response in the donor animal's immune system and which results in the production of certain antibodies at desired levels in the donor animals colostrum. The colostrum is collected from the donor animal while the animal is giving birth and then processed, e.g., by sterilizing and filtering, for administration to canines. The colostrum may be administered to mammals at any age from birth to advanced age and may be administered orally and/or intravenously.

The disclosure will discuss primarily the treatment of canines although it is possible to adapt the various methods and compositions discussed herein to the treatment of other types of mammals including, for example, horses, pigs, felines, and humans. The donor animal is typically a different species of mammal than the mammal that is to be treated although not necessarily. The donor animal may be selected for various reasons including, for example, the types of antibodies that are known to be produced by the animal, the ability of the animal to produce colostrum, the amount of colostrum produced, the feasibility of maintaining the donor animal(s), the difficulties faced in challenging the animal and collecting the colostrum, to name a few.

Colostrum is a form of milk produced by the mammary glands of mammals in late pregnancy, and is the first milk produced after birth. Colostrum is rich in specific anti-microbial factors, such as antibodies, or immunoglobulins (e.g., IgG, IgA, IgE), and non-specific antimicrobial factors, such as lactoferrin. Immunoglobulins, also known as antibodies, in colostrum are intended to protect newborns against infection. Lactoferrin provides antibacterial protection to newborns and also controls the level of free iron in blood and external secretions to restrict its use by pathogenic agents. Colostrum also contains large quantities of fat-soluble vitamins that do not cross the placenta, such as vitamins A, D, and E, which are important in bone development and the immune of inflammatory response as well as being lower in fat and higher in protein than ordinary milk.

Maternal antibodies generally do not cross the placenta of some animals or are only partially able to transfer to the fetus during pregnancy. Therefore, newborn mammals depend on intestinal absorption of ingested antibodies from the colostrum to provide protection against disease and infection for the first weeks of life. Colostrum production as well as the newborns ability to absorb colostrum is only for a limited duration after birth. Therefore, colostrum collected from donor animals is collected as soon as possible after the animal has given birth. According to one embodiment, the colostrum produced by the donor animal during the first two days (e.g., approximately 48 hours) after birth is collected. The donor animal selected to produce the colostrum can be any mammal that is capable of producing useful antibodies for treatment. In the discussion below, goats are used as the colostrum donor animals for the canine treatment. However, it is contemplated that other animals, such as cows and sheep, could also be used to provide the donor colostrum.

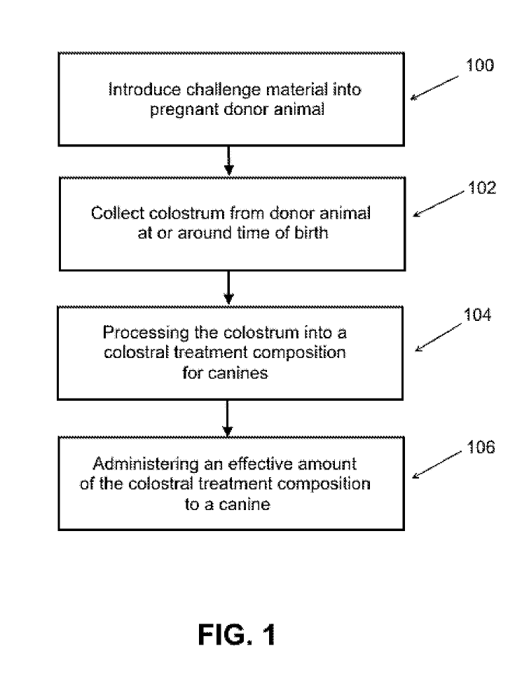

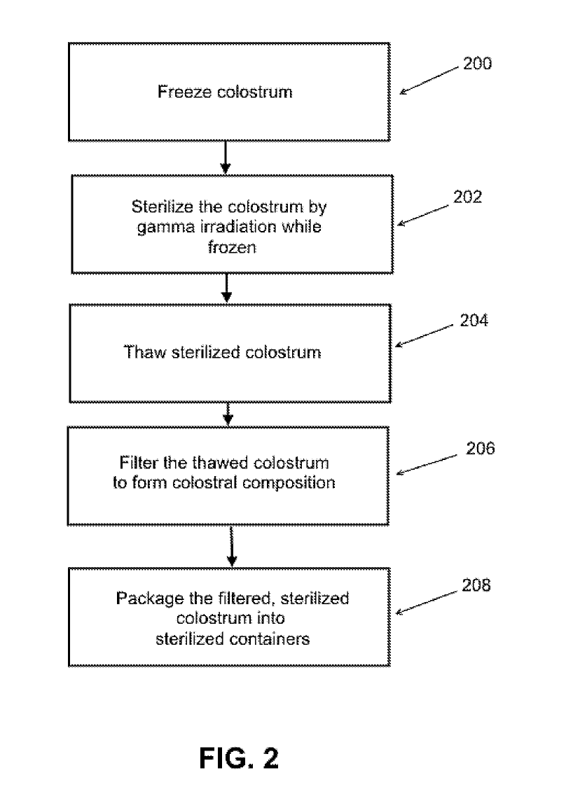

A flowchart of a method of producing a treatment composition and using the composition to treat mammals, e.g., canines, is depicted in FIGS. 1 and 2. According to the FIG. 1, the donor animal is challenged during pregnancy to enhance certain properties of the colostrum produced by the animal (block 100). As used herein, the term “challenged” refers to the controlled introduction of one or more challenge materials into the donor animal's system. Challenge materials comprise one or more antigens or pathogens, such as viral agents, bacterial agents, toxins, parasites, and the like, which are capable of causing an immune response in the donor animal which results in the production of certain antibodies and/or increased levels of antibodies and other agents in the donor animal's colostrum.

The challenge materials are selected to induce an immune response in the donor animal which results in the generation of at least one type of antibody that is capable of fighting at least one type of canine pathogen and toxins, such as, for example, canine parvovirus. Antibodies fight infection by binding with the molecules of pathogens and toxins which cause the infection. The binding neutralizes the molecules causing the infection and/or marks the molecules for destruction or elimination by other cells of the immune system. The antibodies generated in response to a challenge are capable of identifying and binding with molecules which elicit the same or a similar immune response as the challenge. The ability of an antibody to identify and bind a specific type of pathogen or toxin is referred to as the “binding specificity” of the antibody. Accordingly, the challenge materials are selected to induce an immune response in the donor animal which results in the generation of at least one type of antibody having a binding specificity for at least one canine pathogen or toxin.

The challenge material(s) is/are introduced in a manner intended to increase the immune response in the donor animal to a level that is greater than the immune response would be to a natural or normal challenge. This type of challenge or immunization is also referred to as hyperimmunization and results in the production of a greater-than-normal number of antibodies and/or the production of certain antibodies that may not otherwise be produced. Such a response may be induced, for example, by increasing the dose of the antigen(s) in a challenge material that is already given to the donor animal, by using an adjuvant to modify the effect of the antigen(s), and/or by introducing challenge materials that the donor animal would otherwise not be exposed to.

The challenge material may comprise one or more agents, or antigens, that resemble disease-causing microorganisms. The challenge material may be made from weakened or killed forms of the microorganism, its toxins or one of its surface proteins that can be more easily fought and defeated by the body's immune system. The donor animal may be challenged with materials which are normally given to that type of animal to protect against disease. Goats, for example, receive vaccinations against clostridial diseases enterotoxemia and tetanus (i.e., CDT vaccine), pneumonia caused by Pasteurella multocida or Mannheimia haemolytica, leptospirosis, and rabies, and possibly some others.

Alternatively or in addition to the normal challenges administered to the donor animal, the challenge material may comprise one or more agents, or antigens, that resemble disease-causing microorganisms which afflict mammals, such as, for example, canine parvovirus, Escherichia coli (E. Coli), and numerous other bacterial and viral agents.

The donor animal may be challenged at any suitable time and/or suitable number of times while pregnant and prior to giving birth by infusion directly into the udder. The number of times and the timing of the challenges can vary depending on the animal species, the type of challenge, and the desired immune response. One, two, or more challenges may be performed if desired. Each challenge could use the same challenge material with the first challenge being the primary challenge and the additional challenges comprising boosters. Different challenge materials may also be used at different times.

In the embodiment of the method depicted in FIG. 1, goats are used as the donor animal. A first challenge is performed at around four weeks prior to the goat's expected delivery date. Although not required, a second challenge may be performed, e.g., at around two weeks prior to giving birth, to increase the colostral response. A number of different challenge protocols and strategies are possible and contemplated within the scope of this disclosure with the end result being that a supercharged colostrum is produced which has a higher level, and preferably a significantly higher level, of antibodies than would be produced under normal circumstances and/or which produces certain specific antibodies which would otherwise not be produced.

The challenge material may be introduced into the donor animal in any suitable manner that is capable of eliciting the desired immune response in the colostrum. In one embodiment, the challenge material is introduced directly to the donor animal's mammary glands, or udders, which helps ensure that the desired antibodies are produced and concentrated in the donor animal's colostrum. For example, the challenge material may be infused up the mammary duct in the udder of the donor animal using a smooth cannula.

Referring to flowchart of FIG. 1, the colostrum is collected at or around the time the donor animal is giving birth (block 102). The colostrum can be collected in any suitable manner, such as by hand or by using a colostrum collection device. Because the colostrum from the donor animal will not be available for the newborn animal, a supply of colostrum, e.g., taken from other animals of the same type, should be made ready so it can be provided to the newborn at birth in place of the colostrum collected for the canine treatment.

In one embodiment, the colostrum produced by the donor animal during the first 2 days, or approximately 48 hours, is collected. In alternative embodiments, the colostrum may be collected during a smaller time period after birth, e.g., the first 6, 12, or 18 hours, or a longer period, e.g., 3 or 4 days. The quality and concentration of antibodies in the colostrum as well as the type, age, and condition of the donor animal are examples of factors that can be taken into consideration to determine optimal times and durations for collecting colostrum from a particular donor animal.

After the colostrum has been collected, the colostrum is processed into a colostral treatment composition that is suitable for administration to canines as part of a treatment strategy (block 104). An effective amount of the colostral treatment composition is then administered to a canine to treat and/or prevent at least one canine pathogen, such as canine parvovirus (block 106). In one embodiment, the colostral composition is administered orally to the mammal to be treated, which in this case is canines, at any stage from newborn to adult. In alternative embodiments, the colostral composition may be administered to other types of mammals, including pigs, horses and humans. For example, a colostral composition may be administered to treat against agents, such as TGE and E.Coli. Colostral compositions may be administered to horses, e.g., as foals, as treatments against agents, such as R. equi. Colostral compositions may be administered to felines as treatments against agents, such as feline leukemia. Colostral compositions may also be administered to humans as treatments against agents, such as C. diff and Epstein-Barr. These are only examples of the possibilities for using colostral compositions to treat bacterial and viral agents in different types of mammals. The use of colostral compositions, such as described herein, for treating mammals is not limited to the types of mammals and agents described herein. The disclosure is intended to encompass the use of colostral compositions, such as described herein, to treat mammals against substantially any kind of bacteria agent, viral agent, toxin, or other type of pathogen.

FIG. 2 depicts a flowchart of a method of processing the colostrum from donor animals into a colostral treatment composition for administration to canines. According to FIG. 2, the colostrum is refrigerated or frozen as soon as possible to prevent bacterial growth and to preserve the antibodies in the colostrum (block 200). While frozen, the collected colostrum can be stored and transported as needed until further processing can take place. Sterilization is performed to eliminate microbial life from the colostrum, such as fungi, bacteria, viruses, spores, etc. The sterilization process that is used must be capable of sterilizing the colostrum without (or with minimal) denaturing or otherwise damaging the colostrum.

In one embodiment, the colostrum is sterilized by gamma-irradiation while the colostrum is maintained in a frozen state (block 202). In this embodiment, the colostrum is frozen to a hard freeze condition so that the colostrum remains frozen during the irradiation. The colostrum is subjected to sufficient gamma irradiation that the material is sterilized, but is not denatured. In one embodiment, the gamma irradiation that was found to be sufficient to sterilize the frozen colostrum without denaturing the colostrum was between 1.0 to 4.5 Mrad. In alternative embodiments, other methods of sterilization may be used as long as they are capable of sterilizing the colostrum while preventing or minimizing denaturing.

The sterilized colostrum is thawed so it can be filtered and then packaged into sterile containers (block 204). The colostrum should be thawed slowly in order to minimize the possibility of denaturing the colostrum and damaging the antibodies. Any suitable method of thawing the colostrum may be used. For example, the frozen colostrum may be thawed by placing the colostrum in warmer refrigeration units. Methods of thawing that could result in uneven thawing and the formation of “hot spots” within the colostrum should be avoided.

Once thawed, the colostrum is filtered using a sterile filtration process to remove cellular matter and particulates, such as aggregates of lipids, proteins, and other materials, which may interfere with absorption and/or result in sterile abscesses (block 206). Any suitable method of sterile filtration, type of filter media, and rating (e.g., micron rating) may be used. A single filtration step or multiple filtration steps may be used. For example, in one embodiment, the thawed colostrum is filtered through a series of successively smaller rated filters, e.g., starting with a 10 micron filter, followed by a 5 micron filter, and finishing with a 3 micron filter.

The colostrum from different donor animals (of the same type, e.g., goats) may be combined at some point during processing, such as during or after filtration. In addition, one or more additives may be added to the colostral composition after filtration to improve or enhance characteristics or performance. For example, flavoring agents may be added to improve taste for oral administration. The filtered colostrum is then packaged in sterile containers and refrigerated until it is administered (block 208). The size and type of containers used can vary depending on the method of administration, intended use, intended dosage, and the like. For example, the processed may be packaged in small containers, such as bottles or syringes, in single dose or multiple dose sizes which allow the colostrum to be easily handled and dispensed.

The colostral composition may be administered to mammals orally at any stage from newborn to adult. It is also contemplated that the colostral composition be processed for administration via subcutaneous injection. The composition may be administered at the first sign of infection. For example, the colostral composition may be administered to canines at the first sign of parvovirus symptoms. In addition, any dogs that have been in contact with a possibly infected animal may also be administered the colostral composition (and quarantined) as a preemptive measure. For example, in the case of newborn litters, if one litter mate shows signs of parvovirus infection, the entire litter may be treated.

Any suitable dosage size of the colostral composition may be used. The age and size of the mammal may be factors in determining the appropriate dosage. In one embodiment, dosage size of the colostral composition is from 1 ml to 5 ml for newborn puppies and from 5 ml to 50 ml for older dogs. The composition may be delivered into the mouth of the canine, for example, when feasible based on the condition of the dog and any symptoms being exhibited and preferably after experiencing any vomiting or diarrhea episode.

In one embodiment, the colostrum treatment composition is administered orally to newborn mammals, such as puppies, foals, pigs, and humans, soon after birth, e.g., within the first few 12 hours or so. During this time, the mammal still has the ability to absorb antibodies from ingested colostrum through the intestinal walls and into the bloodstream. The antibodies absorbed in this manner from the colostrum treatment will confer a passive immunity onto the treated mammal in a manner similar to the passive immunity conferred by the normal colostrum received from the mother. The advantage in this case is that the amount and types of antibodies introduced into the animal's bloodstream by the colostrum treatment composition are capable of providing a broader level of immunity and protection against disease and infection than would otherwise be the case.

The colostrum treatment composition may be administered to mammals in all stages of life, from newborn to adult, for treating active infections before or after they are symptomatic. When administered to animals after window of absorption has closed and they are no longer able to absorb antibodies into the bloodstream via the intestinal walls, the antibodies will not be able to confer passive immunity onto the treated animal. However, the antibodies in the colostrum treatment composition will still act directly on organisms within the lumen of the gut. Therefore, the colostrum treatment composition can be used to directly combat infections within the gastrointestinal tract. This is particularly beneficial against certain infections, such as canine parvovirus, which cause severe inflammation of the walls of the stomach and intestines.

It is also contemplated that the colostral composition be administered at various other times and frequencies depending on the type of animal being treated and the type of pathogen being treated against. For example, the colostral composition may be administered on a daily, weekly, biweekly, or monthly basis or in accordance with any other suitable frequency or timing. When administered on a repeating schedule, the treatment may continue until the infection has been eradicated and possibly for some duration after. Determining to continue treatment with the colostral composition may be made based on appropriate diagnostic markers for the infection being treated. As one example, the colostral composition may be administered to human (infants) daily as a treatment for Epstein-Barr. Markers used to determine the efficacy of the treatment include reduced chronic fatigue and fibromyalgia.

The treatment composition and method described above have been tested on canines to determine the efficacy of the treatment against various canine infections as well as effective protocols for the treatment. Colostral treatment compositions were produced by challenging pregnant donor animals, primarily goats, by infusing challenge materials directly into the udder at least one, e.g., at four weeks, before birthing. The colostrum was collected during birthing and processed as described above to produce a colostral treatment composition. The composition was administered to canines in response to exhibiting symptoms or possible exposure to various infections, including canine parvovirus, TGE, E. coli, R. equi, C. diff, and Epstein-Barr.

To test the efficacy of the treatment methods describe herein, colostral compositions were collected from donor animals and administered to mammals, as described above. The colostral composition was administered at three veterinary clinics to puppies as a treatment for parvovirus infection. The treatment was administered to puppies at the onset of any signs of infection or to puppies that were exposed to other infected animals. A survival rate of approximately 90% was reported for canines infected with canine parvovirus that had been treated with the colostral composition. A colostral composition was generated with R. equi antibodies to administer to foals. In one test, 17 horses that were treated with the colostral composition did not express R. equi symptoms while 4 out of 9 horses that were not treated with the colostral composition exhibited signs of R. equi infection and required aggressive treatment.

While the disclosure has been illustrated and described in detail in the drawings and foregoing description, the same should be considered as illustrative and not restrictive in character. It is understood that only the preferred embodiments have been presented and that all changes, modifications and further applications that come within the spirit of the disclosure are desired to be protected.

1. A method of treating and/or preventing infectious disease in mammals, the method comprising:
collecting a colostral composition from a donor animal, the colostral composition including at least one type of antibody having a binding specificity for at least one pathogen, the at least one type of antibody being generated by the donor mammal in response to being challenged with at least one challenge material,
freezing the collected colostral composition;
sterilizing the frozen colostral composition by gamma irradiation;
thawing the sterilized colostral composition; and
administering an effective amount of the thawed colostral composition to a mammal.

2-3. (canceled)

4. The method of claim 1, wherein the donor species is immunized to generate the antibodies.

5. The method of claim 1, wherein the non-canine donor species is challenged during pregnancy.

6. The method of claim 1, wherein the non-canine donor species is selected from a group comprising cow, goat, and sheep.

7. The method of claim 1, wherein the colostral composition is administered orally to the mammal.

8. The method of claim 1, wherein the colostral composition is administered to the mammal within approximately 48 hours of birth.

9. The method of claim 1, wherein the mammal is a canine, and
wherein the colostral composition is administered to the canine in response to being infected by a canine pathogen.

10. The method of claim 9, wherein the canine pathogen comprises canine parvovirus.

11. A method of treating and/or preventing infectious disease in mammals, the method comprising:
introducing at least one challenge material into a pregnant, donor animal, the at least one challenge material being selected to cause at least one type of antibody to be produced in the donor animal's colostrum, the at least one type of antibody having a binding specificity for at least one pathogen;
collecting colostrum from the donor animal;
freezing the collected colostrum;
sterilizing the frozen colostrum by gamma irradiation;
processing the sterilized colostrum into a colostral composition for administration to a mammal; and
administering the colostral composition to the mammal.

12-14. (canceled)

15. The method of claim 11, wherein the processing further comprises:
allowing the sterilized colostrum to thaw; and
filtering the thawed, sterilized colostrum to produce the colostral composition.

16. The method of claim 15, wherein the introduction of the at least one challenge material further comprises:
immunizing the donor animal to generate the at least one type of antibody.

17. The method of claim 16, wherein the mammal is a canine, and
wherein the colostral composition is administered orally to the canine.

18. The method of claim 17, wherein the colostral composition is administered to a puppy within approximately 48 hours of birth.

19. The method of claim 17, wherein the colostral composition is administered to the canine in response to being infected by a canine pathogen.

20. The method of claim 19, wherein the canine pathogen comprises canine parvovirus.

21. The method of claim 11, wherein introducing the challenge material further comprises:
introducing the challenge material via the donor animal's udder.
